(19) United States
(12) Patent Application Publication
(10) Pub. No.: US 2026/0157737 A1
(43) Pub. Date: Jun. 11, 2026

(54) QUADRICEP TENDON HARVESTOR

(71) Applicant: Anup A. Shah, Phoenix, AZ (US)

(72) Inventor: Anup A. Shah, Phoenix, AZ (US)

(21) Appl. No.: 19/414,136

(22) Filed: Dec. 9, 2025

Related U.S. Application Data
(60) Provisional application No. 63/729,948, filed on Dec. 9, 2024.

(51) Int. Cl. A61B 17/00; A61B 17/3205

(52) U.S. Cl. CPC A61B 17/00008; A61B 17/3205; A61B 2017/00017

(57) ABSTRACT

A quadricep tendon harvestor for harvesting a quadricep tendon from a patient, comprising: an advancement mechanism; a hood, the hood advanced by the advancement mechanism in a superior direction along the quadricep tendon of the patient; an outrigger, the outrigger stabilizing the quadricep tendon harvestor relative to the patient; a cutting element, the cutting element disposed on the inferior surface of the hood, whereby the cutting element cuts the patient's quadricep tendon, thereby allowing harvesting of the quadricep tendon.

### CROSS-REFERENCE TO RELATED APPLICATIONS

Benefit of priority to the following three United States Provisional Patent Applications is claimed: (i) Application Ser. No. 63/729,948, filed on Dec. 9, 2024, and titled “Quadricep Tendon Harvestor,” (ii) U.S. Provisional Patent Application Ser. No. 63/746,057, filed on Mar. 19, 2024, and titled “Quadricep Tendon Harvestor,” and (iii) Application Ser. No. 63/793,478, filed on Apr. 23, 2025, and titled “Quadricep Tendon Harvestor” are claimed.

### STATEMENT REGARDING FEDERALLY SPONSORED RESEARCH

Not Applicable.

### BACKGROUND OF THE INVENTION

The anterior cruciate ligament (ACL) is one of four bands of tissue that stabilize the human knee. Specifically, the ACL prevents the shinbone (tibia) from sliding in front of the thighbone (femur) and keeps the knee from rotating. When this critical ligament tears; the tear may be partial or complete, in either case, surgery is often needed. This need is particularly acute when the ACL is completely torn, i.e., completely separated. The process is generally referred to as ACL reconstruction.

A variety of methods for repairing an ACL have been developed since the 1970's. More recently, ACL reconstruction has utilized the quadriceps tendon as an autograft in anterior cruciate ligament reconstruction (ACLR). Use of a patient's own quadriceps tendon is referred to as an “autograft.” Autographed tissue is preferable because it has been found to lessen the chance of infection and promote healing.

FIG. 1 illustrates a human right knee and illustrates that the quadriceps tendon works with the quadricep muscle to extend and retract the leg. Each of the four sections of the quadricep muscle attach to the shin (tibia) via the patella (knee cap). In colloquial English, the patella is known as the knee cap. As also illustrated in FIG. 1, the patella tendon, sometimes referred to as the patella ligament, fastens to the tibial tuberosity of the tibia. The anterior cruciate ligament, commonly referred to as the ACL, is one of the cruciate ligaments of the human knee. An ACL tear is among the most common knee injuries with experts believing that more than 100,000 such injuries occurring yearling the United States. The tear can be a compete severing of the ACL or a partial tear. In many cases, the torn ACL is completely removed and replaced with a piece of quadricep tendon from the patient (known as an autograft) or from a donor (known as an allograft). In either situation, the ACL surgery requires harvest of a quadricep tendon to replace the patient's torn ACL.

Current techniques of quadricep tendon harvesting are an improvement from the original, almost forty year old, techniques. However, difficulty with direction (angulation and medial lateral direction) can potentially cause premature tendon transection, resulting in shorter and thinner graft than desired. Additionally, current techniques have concerns with assessing depth through inferior and superior tilting of the existing quad harvestor as well as assessing the medial or lateralized direction. The existing technique has attempted to remedy this concern by using a clear (see through) section of the harvesting tool, but even with this modification, requires that both the vertical and horizontal planes need to be meticulously observed to avoid premature transection of the graft. In addition, with smaller incisions and advocates for a transverse distal incision, viewing the proximal tapered portion of the quadricep tendon become challenging.

### SUMMARY OF THE INVENTION

A quadricep tendon harvestor for harvesting a quadricep tendon from a patient, comprising: an advancement mechanism; a hood, the hood advanced by the advancement mechanism in a superior direction along the quadricep tendon of the patient; an outrigger, the outrigger stabilizing the quadricep tendon harvestor relative to the patient; a cutting element, the cutting element disposed on the inferior surface of the hood, whereby the cutting element cuts the patient's quadricep tendon, thereby allowing harvesting of the quadricep tendon.

These and other embodiments will be more fully appreciated from the description below.

### BRIEF DESCRIPTION OF THE DRAWINGS

FIG. 1 illustrates a human right knee and reveals that the quadriceps tendon works with the quadricep muscle to extend and retract the leg.

FIG. 2 illustrates an environmental left side view of the first embodiment of the quadricep harvestor in its unextended position. The right side view is a mirror image.

FIG. 3 illustrates a left side view of the first embodiment of the quadricep harvestor in its extended position. The right side view is a mirror image.

FIG. 4 illustrates a sectional view of the cutting element of the quadricep harvestor.

FIG. 5 illustrates an oblique head on view of the hood and the cutting element of the quadricep harvestor.

FIG. 6 illustrates an environmental view of the quadricep tendon.

FIG. 7 illustrates a perspective environmental view of a second embodiment of the quadricep harvestor before incising the tendon. The opposing side is a mirror image.

FIG. 8 illustrates a perspective environmental view of a second embodiment of the quadricep harvestor after incising the tendon. The opposing side is a mirror image.

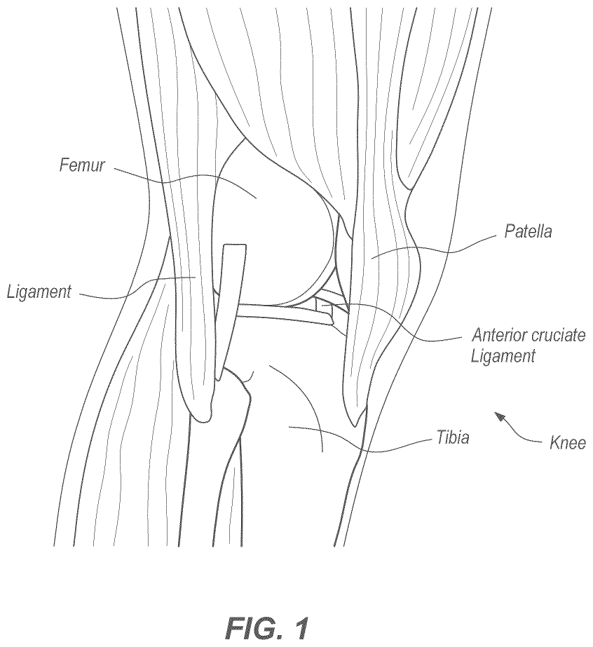

### DESCRIPTION OF THE PREFERRED EMBODIMENTS

Corresponding reference numbers indicate corresponding parts throughout the several views of the drawings and specification.

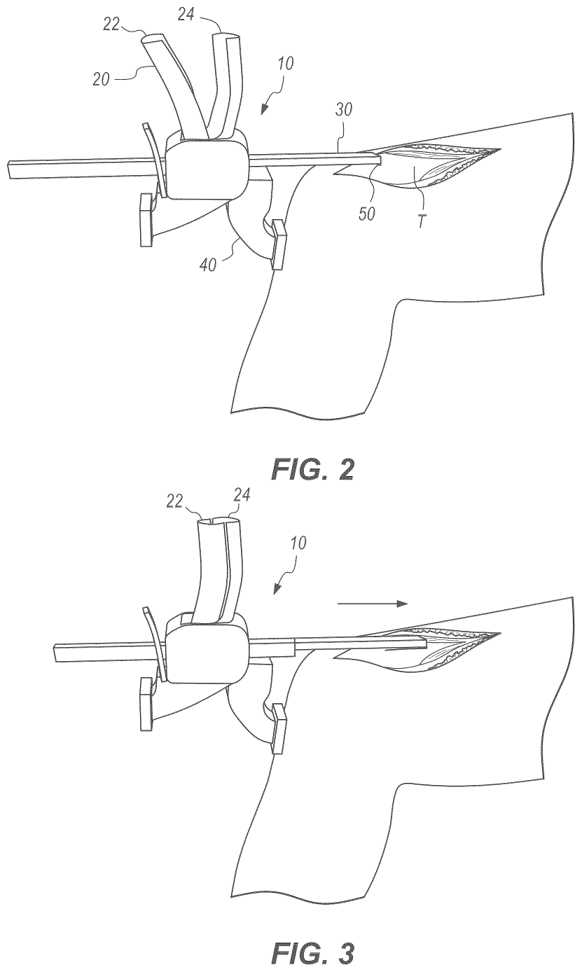

FIG. 2 illustrates an environmental view of the first embodiment of the quadricep harvestor 10. FIG. 2 further illustrates an advancing mechanism 20, a hood 30, an outrigger 40, and a cutting element 50. As further illustrated in FIG. 2, when trigger 22 is compressed, cutting element 50 is advanced along the superior surface of tendon T. In order to mitigate overadvancement of advancement mechanism 20, trigger stop 24 is positioned to prevent further compression of trigger 22. It should be understood that a number of linear motion mechanisms, such as mechanical, electrical, hydraulic, or pneumatic cylinders may be used. In addition, a belt-driven linear actuator could also be used or a screw-driven linear actuator could also be used. It is believed that the screw-driven actuator is preferably a lead screw or a ball screw. Also in addition, a rack and pinion linear mechanism using a gear mechanism could also be used. Similarly, an electrical actuator could use an electric motor to create linear motion of cutting element 50. In addition, a solenoid could be used to advance cutting element 50. In addition, a piezoelectric linear motion mechanism could also be used. It is also believed that electrical linear actuators are preferred in medical applications, such as the present situation, because of their precise movement control. Other types of mechanisms could also be used to advance cutting element 50.

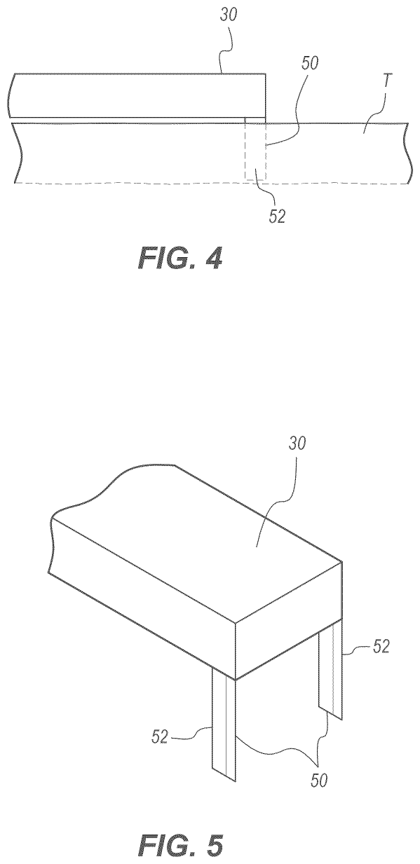

FIGS. 2 and 3 illustrate cutting element 50. Preferably, as illustrated in FIG. 4, cutting element 50 is opposing knife blades 52.

Most preferably, as illustrated in FIGS. 4 and 5, knife blades 52 are on opposite sides of hood 30 thereby cutting tendon T to harvest it for use in an ACL repair. FIGS. 2 and 3 illustrates outrigger 40. Outrigger 40 stabilizes quadricep harvestor 10 and minimizes relative motion between the patient, and particularly the patient's leg, and quadricep harvestor 10. Preferably, outrigger 40 has a flat surface that, preferably, is sized to touch and rest against the patient's knee cap and thereby minimizes or eliminates relative movement between the patient and quadricep harvestor 10. Ideally, the relative motion will be negligible or zero.

FIGS. 4 and 5 illustrate a view of cutting element 50 disposed on the inferior side of hood 30. FIG. 4 further illustrates knife blade 52 incising tendon T.

FIG. 3 illustrates a view of the first embodiment of the quadricep harvestor 10 after it has been advanced along Tendon T in preparation for harvesting.

FIGS. 2 and 3 illustrate cutting element 50 advanced by advancement mechanism 20 along the superior surface of tendon and thereby incising the tendon for harvesting. Phrased differently, FIG. 2 illustrates quadricep harvestor 10 at its initial position and FIG. 3 illustrates quadricep harvestor 10 after it has been advanced along tendon T to incise (cut) tendon T for harvesting.

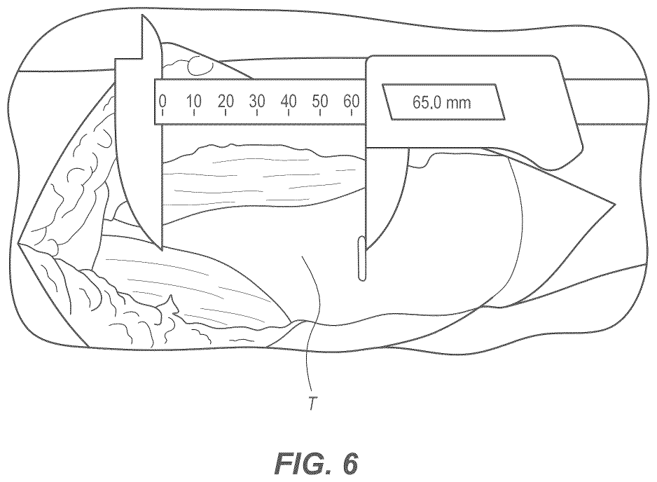

FIG. 6 illustrates the exposed quadricep tendon T and further illustrates the typical length of a harvested section of the tendon T is 65 millimeters.

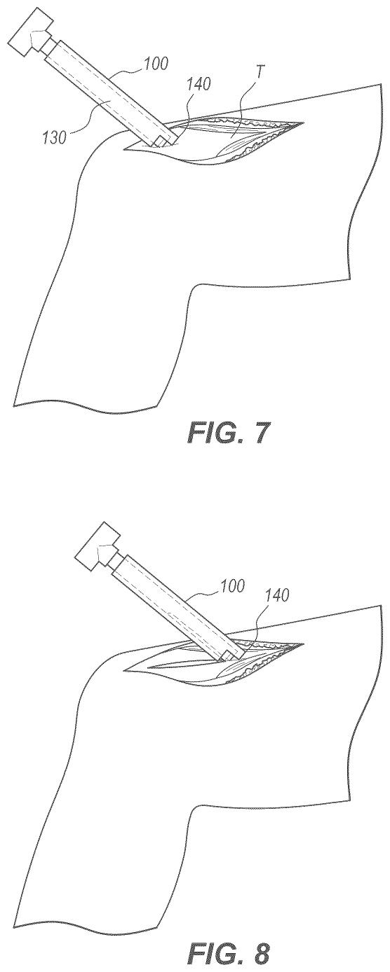

FIG. 7 illustrates a second embodiment of the quadricep harvestor 100. Quadricep harvestor 100 includes shaft 130 and tab 140. Quadricep harvestor 100 is used to manually incise and harvest tendon T. Shaft 130 includes a cutter surface to incise tendon T for harvesting. Preferably, a cutter surface 132 is opposing cutters which thereby incise (cut) a section of tendon T for use in an ACLR procedure. Tab 140 stabilizes quadricep harvestor 100 relative to tendon T. Phrased differently, tab 140 thereby minimizes or eliminates relative movement between the patient and quadricep harvestor 10. Ideally, the relative motion will be negligible or zero. It is believed that the use of tab 140 is an improvement of the current prior art harvestors in current use. When the terms “distal” and “proximal” are used in reference to quadricep harvestor 100, the user will hold the proximal section and the cutter surface will be distal, i.e., tab 140 is located at the distal end of shaft 130.

While the invention has been illustrated and described in detail in the drawings and description, the same is to be considered as an illustration and is not limited to the exact embodiments shown and described. All equivalents, changes and modifications that come within the spirit of the invention are also protected by the claims that are set forth below.

I claim:

1. A quadricep tendon harvestor for harvesting a quadricep tendon from a patient, comprising:
an advancement mechanism;
a hood, the hood advanced by the advancement mechanism in a superior direction along the quadricep tendon of the patient;
an outrigger, the outrigger stabilizing the quadricep tendon harvestor relative to the patient;
a cutting element, the cutting element disposed on the inferior surface of the hood, whereby the cutting element cuts the patient's quadricep tendon, thereby allowing harvesting of the quadricep tendon.

2. The quadricep tendon harvestor of claim 1, wherein the advancement mechanism is a linear motion mechanism.

3. The quadricep tendon harvestor of claim of claim 2, wherein the linear advancement mechanism is a mechanical, electrical, hydraulic, or pneumatic.

4. The quadricep tendon harvestor of claim of claim 3, wherein the linear advancement mechanism is an electrical actuator.

5. The quadricep tendon harvestor of claim of claim 4, wherein the electrical actuator of claim 4 is a solenoid.

6. The quadricep tendon harvestor of claim 3, wherein the linear actuator is belt-driven, screw-driven, or a rack and pinion mechanism.

7. The quadricep tendon harvestor of claim 3, wherein the hydraulic linear actuator is actuated by the trigger.

8. The quadricep tendon harvestor of claim 3, wherein the pneumatic linear actuator is actuated by a trigger.

9. The quadricep tendon harvestor of claim 6, wherein the screw-driven actuator employs a lead screw or a ball screw.

10. The quadricep tendon harvestor of claim 4, wherein the electrical actuator is a piezoelectric linear motion mechanism.

11. The quadricep tendon harvestor of claim 1, wherein the cutting element is a pair of opposing knife blades.

12. The quadricep tendon harvestor of claim 1, wherein the hood and the stabilizer are positioned to maintain the relative position of the cutting element relative to the patient's tendon and thereby minimizing or eliminating relative movement between the patient and quadricep harvestor.

13. A quadricep harvestor for harvesting a quadricep tendon from a patient, comprising, comprising:
a shaft;
a tab disposed on the distal end of the shaft, the tab, whereby the tab contacts the patient's tendon and thereby stabilizes quadricep harvestor relative to tendon; and,
a cutting element, the cutting element disposed proximate to the tab,
whereby the cutting element cuts the patient's quadricep tendon, thereby allowing harvesting of the quadricep tendon.

14. The quadricep tendon harvestor of claim 13, wherein the cutting element is a pair of opposing knife blades disposed on the distal end of the quadricep harvestor.

15. The quadricep harvestor of claim 13, wherein the cutting element is disposed in the interior of the quadricep harvestor.
